(19) United States
(12) Patent Application Publication
(10) Pub. No.: US 2011/0023156 A1
(43) Pub. Date: Jan. 27, 2011

(54) FELINE GENOME EDITING WITH ZINC FINGER NUCLEASES

(75) Inventors: Joseph Bedell, St. Louis, MO (US); Gregory Davis, St. Louis, MO (US); Shondra Miller, St. Louis, MO (US); Brian Buntaine, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,219

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/027; C12N 5/071; C12N 5/10; C12N 9/16; C07H 21/04

(52) U.S. Cl. 800/14; 435/351; 435/196; 536/23.5

(57) ABSTRACT

The present invention provides a genetically modified feline or cell comprising at least one edited chromosomal sequence. In particular, the chromosomal sequence is edited using a zinc finger nuclease-mediated editing process. The disclosure also provides zinc finger nucleases that target specific chromosomal sequences in the feline genome.

### CROSS REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified felines or feline cells comprising at least one edited chromosomal sequence. In particular, the invention relates to the use of targeted zinc finger nucleases to edit chromosomal sequences in the feline.

### BACKGROUND OF THE INVENTION

The domestic cat or house cat is the most popular pet in the world, with a worldwide population in excess of 500 million. While 41 different breeds of cats are recognized the Cat Fanciers' Association, all cats come in a variety of colors, coat patterns, and hair lengths. Coat patterns include bicolor, tabby, tortoiseshell, and colorpoint, with cats having short hair, long hair, or curly hair. Humans have been breeding cats for years to obtain desirable coat colors, coat patterns, and/or hair growth.

Although cats are desirable pets, some humans are allergic to cats. The primary allergen is Felis domesticus 1 (Fel d1), which is produced by cat salivary and sebaceous glands. Thus, hypoallergenic cats with reduced levels of Fel d1 are desirable, and nonallergenic cats with no Fel d 1 would be even more desirable. Another drawback associated with having cats as pets is the problem of cat urine odor and associated territorial (or spraying) behaviors. Cauxin is a kidney-specific carboxylase that generates the pheromone precursor felinine, which is the major odor producing compound in urine. Since cauxin is not produced in kittens until about 2-3 months of age, it likely is not an essential gene. Knockout of the cauxin gene, therefore, may eliminate cat urine odor problems and certain undesirable behaviors. Thus, there is a need for improved methods of knocking out genes coding allergenic proteins or protein involved in urine odor, as well as means for modifying other genes involved in desirable cat phenotypes.

### SUMMARY OF THE INVENTION

Among the various aspects of the present disclosure is the provision of methods for modifying feline chromosomal sequences through zinc finger nuclease-mediated genome editing.

One aspect of the present disclosure encompasses a genetically modified feline comprising at least one edited chromosomal sequence.

A further aspect provides a feline embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

Another aspect provides a genetically modified feline cell comprising at least one edited chromosomal sequence.

Yet another aspect provides a zinc finger nuclease comprising a zinc finger DNA binding domain and a cleavage domain. The zinc finger nuclease has an amino acid sequence that has at least about 80% sequence identity to a sequence chosen from SEQ ID NOs:1, 2, 3, 4, 5, 6, 7, and 8, or the zinc finger DNA binding domain helices have an amino acid sequence that is at least about 80% identical to a sequence chosen from SEQ ID NOs:19, 20, 21, 22, 23, 24, 25, and 26.

Another aspect encompasses a nucleic acid sequence that is recognized by a zinc finger nuclease. The nucleic acid sequence has at least about 80% sequence identity with a sequence chosen from SEQ ID NOs:9, 10, 11, 12, 13, 14, 15, 16, 27, 28, 29, 30, 31, 32, 33, and 34.

Other aspects and features of the disclosure are described more thoroughly below.

### DESCRIPTION OF THE FIGURES

FIG. 1 illustrates ZFN-mediated cleavage of SMAD4 in human and feline cells, as detected by a Cel-1 surveyor nuclease assay. G=GFP (no ZFN control). Z=SMAD4 ZFN (191160/19159). Arrows denote cleavage products.

FIG. 2 depicts Cel-1 assays confirming SMAD4 ZFN activity in cat embryos.

FIG. 3 illustrates cleavage of Fel d1 in AKD cells. Presented is Cel-1 screening of the Fel d1 ZFN pair 17, 18 cleavage of chain 1-exon 1.

FIG. 4 illustrates cleavage of Fel d1 chain 1-exon 2 in AKD cells by the Fel d1 ZFN pair 7, 9.

FIG. 5 depicts Cel-1 analysis of the Fel d1 ZFN pair 12/13 cleavage of chain 1-exon 2 in AKD cells.

FIG. 6 illustrates cleavage of Fel d1 locus in cat embryos by ZFN pairs 17, 18 and 12, 13. Lanes 1, 2, 7, and 8 contain samples from individual blastocysts derived from embryos injected with 40 ng/μL of ZFNs. Lane 3 presents a sample from a blastocyst derived an embryo injected with 20 ng/μL of ZFNs. Lanes 4, 9, and 10 contain samples from individual morulas derived from embryos injected with 40 ng/μL of ZFNs. Lane 3 presents a sample from a morula derived an embryo injected with 20 ng/μL of ZFNs. Lane 6 presents a sample from a control blastocyst.

FIG. 7 presents the DNA sequence of an edited Fel d1 locus comprising a 4541 bp deletion (SEQ ID NO:17) between the regions coding for chain 2 and chain 1.

FIG. 8 aligns the edited Fel d1 locus (designated by red dotted line, labeled “sample 5”) comprising the 4541 bp deletion with the sequence of the wild-type Fel d1 locus (SEQ ID NO:18). In the edited sample, the binding site for ZFN 13 is truncated (and the binding sire for ZFN 12 is missing), but the binding site for ZFN pair 17, 18 is intact.

FIG. 9 depicts cleavage of the cauxin locus by cauxin ZFN pair 1/2 (lane 2), ZFN pair 9/10 (lane 4), and ZFN pair 17/18 (lane 5) in AKD cells. Lanes 1 and 3 contain samples from control (GFP) cells.

FIG. 10 illustrates cleavage of the cauxin locus by cauxin ZFN pair 29/30 (lane 2). Lane 2 contains a control (GFP) sample.

### DETAILED DESCRIPTION OF THE INVENTION

The present disclosure provides a genetically modified feline or feline cell comprising at least one edited chromosomal sequence. The edited chromosomal sequence may be (1) inactivated, (2) modified to encode a modified gene product, or (3) comprise an integrated sequence. An inactivated chromosomal sequence is altered such that a functional protein is not made. Thus, a genetically modified feline comprising an inactivated chromosomal sequence may be termed a “knock out” or a “conditional knock out.” Similarly, a genetically modified feline comprising an integrated sequence may be termed a “knock in” or a “conditional knock in.” Furthermore, a genetically modified feline comprising a modified chromosomal sequence may comprise a targeted point mutation(s) or other modification such that an altered protein product is produced. The feline chromosomal sequence generally is edited using a zinc finger nuclease-mediated process. Briefly, the process comprises introducing into a feline embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of generating a genetically modified feline using targeted zinc finger nuclease technology is rapid, precise, and highly efficient.

### (I) Genetically Modified Feline

One aspect of the present disclosure provides a genetically modified feline in which at least one chromosomal sequence is edited. In one embodiment, the edited chromosomal sequence may be inactivated such that the sequence is not transcribed, the coded protein is not produced, or the sequence does not function properly. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. Thus, a chromosomal sequence that is inactivated may be termed a “knockout.” The inactivated chromosomal sequence comprises no exogenously introduced sequence. Feline chromosomal sequences that may be desirable to inactivate or knockout include those coding allergen proteins, as detailed below.

In another embodiment, the edited chromosomal sequence in the genetically modified feline may be modified such that it codes for an altered gene product or the function of the sequence is altered. For example, a chromosomal sequence coding a protein may be modified such that at least one nucleotide is changed and the expressed protein comprises at least one amino acid change, wherein the modified protein gives rise to a phenotypic change in the feline. Alternatively, a chromosomal sequence that functions as a control sequence may be modified such that it is always active or is regulated by an exogenous signal, for example.

In yet another embodiment, the edited chromosomal sequence in the genetically modified feline may comprise an integrated sequence, fusion protein, a microRNA, and the like. The integrated protein coding sequence may be linked to a reporter sequence (the reporter sequence may be linked 5′ or 3′ to the protein coding sequence). An integrated protein coding sequence may be placed under control of an endogenous promoter, may be operably linked to an exogenous promoter, or may be fused in-frame with an endogenous protein coding sequence. Additionally, the integrated sequence may function as a control element. Accordingly, the integrated sequence may be endogenous or exogenous to the feline. An organism or cell comprising such an integrated sequence may be termed “knock in,” and it should be understood that, in an iteration of the feline that no selectable marker is present.

In yet another embodiment, the genetically modified feline may comprise at least one edited chromosomal sequence encoding a protein of interest such that the expression pattern of the protein is altered. For example, regulatory regions controlling the expression of the protein, such as a promoter or transcription factor binding site, may be altered such that the protein of interest is over-produced, or the tissue-specific or temporal expression of the protein is altered, or a combination thereof. Alternatively, the expression pattern of the protein of interest may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence of interest. The genetically modified animal comprising the lox-flanked chromosomal sequence of interest may then be crossed with another genetically modified animal expressing Cre recombinase. Progeny animals comprising the lox-flanked chromosomal sequence and the Cre recombinase are then produced, and the lox-flanked chromosomal sequence encoding a protein of interest is recombined, leading to deletion or inversion of the chromosomal sequence encoding the protein of interest. Expression of Cre recombinase may be temporally and conditionally regulated to effect temporally and conditionally regulated recombination of the chromosomal sequence encoding the protein of interest.

Exemplary examples of feline chromosomal sequences to be edited include those that code for proteins such as allergen proteins, proteins involved in urine odor production, and proteins involved in coat color, coat pattern, and/or hair length. Preferred allergen proteins include Felis domesticus 1 (Fel d1), which is the primary allergen present on cats and is a heterodimer of chain 1 and chain 2 peptides encoded by separate genes in the feline genome. Proteins involved in the production of urine odor include cauxin, which generates the major urinary pheromone felinine. Non-limiting examples of suitable coat color proteins include tyrosinase (TYR), tyrosinase-related protein 1 (TYRP1), augoti signaling protein (ASIP), and melanophilin (MLPH). A non-limiting example of a protein involved in hair length is fibroblast growth factor 5 (FGF5). Those of skill in the art appreciate that many other proteins are involved in coat color, coat patter, and hair length, but their genetic loci have not been determined.

In one embodiment, the genetically modified feline may comprise an edited chromosomal sequence encoding either chain 1 or chain 2 or Fel d1 or both, wherein the edited chromosomal sequence comprises a mutation such that Fel d1 is not produced. The mutation may be a point mutation in which one nucleotide is substituted for another, a deletion mutation in which one or more nucleotides are deleted from the chromosomal sequence, or an insertion mutation in which one or more nucleotides are introduced into the chromosomal sequence. Accordingly, the deletion, insertion, or point mutation may lead to a frame shift or splice site mutation such that premature stop codons are introduced and functional chain 1 and/or chain Fel d1 polypeptides are not made. Accordingly, the Fel d1 chromosomal sequence is inactivated. Thus, a genetically modified feline comprising an inactivated Fel d1 chromosomal sequence may be hypoallergenic or nonallergenic.

In another embodiment, the genetically modified feline may comprise an edited chromosomal sequence encoding TYR, wherein the edited chromosomal sequence comprises at least one modification such that an altered version of TYR is produced. The modification may be a missense mutation in which substitution of one nucleotide for another nucleotide changes the identity of the coded amino acid. A variety of missense mutations and amino acid changes are known in the TYR coding region. Non-limiting examples of suitable amino acid changes include G227W (i.e., glycine at position 227 is changed to tryptophan) and G302R (i.e., glycine at position 302 is changed to arginine). The TYR coding region may be edited to comprise more than one missense mutation such that more than one amino acid is changed. Additionally, the chromosomal region may be modified to have a three nucleotide deletion or insertion such that the expressed TYR protein comprises a single amino acid deletion or insertion, provided such a protein is functional. Those of skill in the art will appreciate that many different modifications are possible in the TYR coding region. The modified TYR coding region may give rise to a temperature sensitive TYR protein. In one embodiment, the genetically modified feline comprising a modified TYR chromosomal region may have a Burmese or Siamese phenotype. In other embodiments, the genetically modified feline comprising a modified TYR chromosomal region may have a different coat color, coat pattern, and/or eye color than a feline in which the TYR chromosomal region is not modified.

In still another embodiment, the genetically modified feline may comprise an edited chromosomal sequence encoding TYRP1, ASIP, MLPH, or combinations thereof. The edited chromosomal sequence may comprise at least one modification such that an altered version of TYRP1, ASIP, or MLPH is produced. The chromosomal sequence may be modified to contain at least one nucleotide change such at the expressed protein comprises at least one amino acid change as detailed above. Alternatively, the edited chromosomal sequence may comprise a mutation such that the sequence is inactivated and no protein is made or a defective protein is made. As detailed above, the mutation may comprise a deletion, an insertion, or a point mutation. The genetically modified feline comprising an edited TYRP1, ASIP, and/or MLPH chromosomal sequence may have a different coat color, coat pattern, and/or eye color than a feline in which said chromosomal region(s) is not edited.

In a further embodiment, the genetically modified feline may comprise an edited chromosomal sequence encoding cauxin, wherein the chromosomal sequence is inactivated such that no cauxin is produced. Suitable mutations are discussed above. The genetically modified feline comprising the inactivated cauxin chromosomal sequence described above generally will not excrete cauxin, felinine, and felinine-related compounds. Furthermore, the genetically modified feline having the inactivated cauxin chromosomal sequence described herein may exhibit reduced or no repetitive spraying and/or territorial behavior.

The present disclosure also encompasses a genetically modified feline comprising any combination of the above described chromosomal alterations. For example, the genetically modified feline may comprise an inactivated Fel d1 and/or cauxin chromosomal sequence, a modified TYR chromosomal sequence, and/or a modified or inactivated TYRP1, ASIP, and/or MLPH chromosomal sequence. In each embodiment described herein, the genetically modified feline does not comprise exogenously introduced transposon sequences.

The genetically modified feline may be heterozygous for the edited chromosomal sequence or sequences. In other embodiments, the genetically modified feline may be homozygous for the edited chromosomal sequence or sequences.

The genetically modified feline may be a member of one of the following genera: Acinonyx, Caracal, Catopuma, Felis, Leopardus, Leptailurus, Lynx, Otocolobus, Pardofelis, Prionailurus, and Profelis. Typically, the feline will be from the genus Felis. An exemplary feline is the domestic cat, Felis catus. As used herein, the term “feline” encompasses embryos, fetuses, newborn kittens, juveniles, and adult feline organisms.

### (II) Genetically Modified Feline Cells

A further aspect of the present disclosure provides genetically modified feline cells or cell lines comprising at least one edited chromosomal sequence. The disclosure also encompasses a lysate of said cells or cell lines. The genetically modified feline cell (or cell line) may be derived from any of the genetically modified felines disclosed herein. Alternatively, the chromosomal sequence may be edited in a feline cell as detailed below.

The feline cell may be any established cell line or a primary cell line that is not yet described. The cell line may be adherent or non-adherent, or the cell line may be grown under conditions that encourage adherent, non-adherent or organotypic growth using standard techniques known to individuals skilled in the art. The feline cell or cell line may be derived from lung (e.g., AKD cell line), kidney (e.g., CRFK cell line), liver, thyroid, fibroblasts, epithelial cells, myoblasts, lymphoblasts, macrophages, tumor cells, and so forth. Additionally, the feline cell or cell line may be a feline stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

Similar to the genetically modified felines, the genetically modified feline cells may be heterozygous or homozygous for the edited chromosomal sequence or sequences.

### (III) Zinc Finger-Mediated Genome Editing

In general, the genetically modified feline or feline cell, as detailed above in sections (I) and (II), respectively, is generated using a zinc finger nuclease-mediated genomic editing process. The process for editing a feline chromosomal sequence comprises: (a) introducing into a feline embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method of genome editing are described in more detail below.

### (a) Nucleic Acid Encoding a Zinc Finger Nuclease

The method comprises, in part, introducing into a feline embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition regions may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:I538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

(iii) Exemplary Zinc Finger Nucleases

Provided herein are zinc finger nucleases that recognize and bind target sequences in the feline Fel d1 chromosomal sequence. In some embodiments, the zinc finger nuclease may have an amino acid sequence that is at least 80% identical to a sequence chosen from SEQ ID NOs:1, 2, 3, 4, 5, 6, 7, and 8. In other embodiments, the zinc finger DNA binding domain of the nuclease may have an amino acid sequence that is at least 80% identical to a sequence chosen from SEQ ID NOs: 19, 20, 21, 22, 23, 24, 25 and 26. In additional embodiments, the sequence identity may be about 81, 82, 83, 84, 85, 86, 87, 88, 89, 90, 91, 92, 93, 94, 95, 96, 97, 98, 99, or 100%.

Moreover, the zinc finger nucleases of the invention may recognize and bind a chromosomal sequence having at least about 80, 81, 82, 83, 84, 85, 86, 87, 88, 89, 90, 91, 92, 93, 94, 95, 96, 97, 98, 99, or 100% sequence identity to a sequence chosen from SEQ ID NOs:9, 10, 11, 12, 13, 14, 15, 16, 27, 28, 29, 30, 31, 32, 33, and 34.

### (b) Optional Exchange Polynucleotide

The method for editing chromosomal sequences may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

The method for editing chromosomal sequences may further comprise introducing at least one donor polynucleotide comprising a sequence for integration into the embryo or cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the feline or it may be an exogenous sequence. The sequence for integration may encode a protein or a non-coding RNA (e.g., a microRNA). Thus, the sequence for integration may be operably linked to an appropriate control sequence or sequences. Alternatively, the sequence for integration may provide a regulatory function. Accordingly, the size of the sequence for integration can and will vary. In general, the sequence for integration may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 20 bp to about 2500 bp. In various embodiments, an upstream or downstream sequence may comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 by to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

### (d) Delivery of Nucleic Acids

To mediate zinc finger nuclease genome editing, at least one nucleic acid molecule encoding a zinc finger nuclease and, optionally, at least one exchange polynucleotide or at least one donor polynucleotide is delivered into the feline embryo or cell. Typically, the embryo is a fertilized one-cell stage embryo.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

### (e) Culturing the Embryo or Cell

The method for editing a chromosomal sequence using a zinc finger nuclease-mediated process further comprises culturing the embryo or cell comprising the introduced nucleic acid(s) to allow expression of the zinc finger nuclease.

An embryo may be cultured in vitro (e.g., in cell culture). Typically, the feline embryo is cultured for a short period of time at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the feline species. Routine optimization may be used, in all cases, to determine the best culture conditions for a particular species of embryo. In some cases, a cell line may be derived from an in vitro-cultured embryo (e.g., an embryonic stem cell line).

Preferably, the feline embryo will be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal generally will comprise the disrupted chromosomal sequence(s) in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is edited.

The genetically modified felines disclosed herein may be crossbred to create animals comprising more than one edited chromosomal sequence or to create animals that are homozygous for one or more edited chromosomal sequences. Those of skill in the art will appreciate that many combinations are possible. Moreover, the genetically modified felines disclosed herein may be crossed with other felines to combine the edited chromosomal sequence with other genetic backgrounds. By way of non-limiting example, suitable genetic backgrounds may include wild-type, natural mutations giving rise to known feline phenotypes, targeted chromosomal integration, non-targeted integrations, etc.

### (IV) Applications

The animals and cells disclosed herein may have several applications. In one embodiment, the genetically modified feline comprising at least one edited chromosomal sequence may exhibit a phenotype desired by humans. For example, inactivation of the chromosomal sequence encoding Fel d1 may result in cats that are hypoallergenic or non-allergenic. In other embodiments, the feline comprising at least one edited chromosomal sequence may be used as a model to study the genetics of coat color, coat pattern, and/or hair growth. Additionally, a feline comprising at least one disrupted chromosomal sequence may be used as a model to study a disease or condition that affects humans or other animals. Non-limiting examples of suitable diseases or conditions include albinism, deafness, skin disorders, hair disorders, and baldness. Additionally, the disclosed feline cells and lysates of said cells may be used for similar research purposes.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of SMAD4 in Cat Cells

Zinc finger nuclease (ZFN)-mediated genome editing was tested in cat cells using a ZFN that binds to the human SMAD4 chromosomal sequence because the DNA binding sites in cat and human are identical. The amino acid sequence and corresponding DNA binding site of SMAD4 ZFN pair (19160/19159) are presented in TABLE 1. Capped, polyadenylated mRNA encoding SMAD4 ZFNs (19160/19159) was produced using known molecular biology techniques. The mRNA was transfected into human K562, feline AKD (lung), and feline CRFK (kidney) cells. Control cells were injected with mRNA encoding GFP.

TABLE 1
SMAD4 ZFNs

| | | SEQ | DNA binding site | SEQ |
|---|---|---|---|---|
| | | ID | (Contact sites in | ID |
| Name | ZFN protein sequence | NO: | uppercase; 5'-3')) | NO: |
| 19159 | VPAAMAERPFQCRICMRNFSR | 1 | ctGCTGTCCTGGCTG | 9 |
| | SDNLARHIRTHTGEKPFACDI | | AGgccctgatgct | |
| | CGRKFAQSSDLRRHTKIHTGG | | | |
| | QRPFQCRICMRNFSRSDTLSQ | | | |
| | HIRTHTGEKPFACDICGRKFA | | | |
| | DRSARTRHTKIHTGEKPFQCR | | | |
| | ICMRKFAQSSDLRRHTKIHLR | | | |
| | GS | | | |
| 19160 | VPAAMAERPFQCRICMRNFSE | 2 | gaATGGATtTACTGG | 10 |
| | RGTLARHIRTHTGEKPFACDI | | TCAGCCagctact | |
| | CGRKFAQSADRTKHTKIHTGG | | | |
| | QRPFQCRICMRNFSRSDHLST | | | |
| | HIRTHTGEKPFACDICGRKFA | | | |
| | DNANRTKHTKIHTGGGGSQKP | | | |
| | FQCRICMRNFSQSSNLARHIR | | | |
| | THTGEKPFACDICGRKFARSD | | | |
| | ALTQHTKIHLRGS | | | |

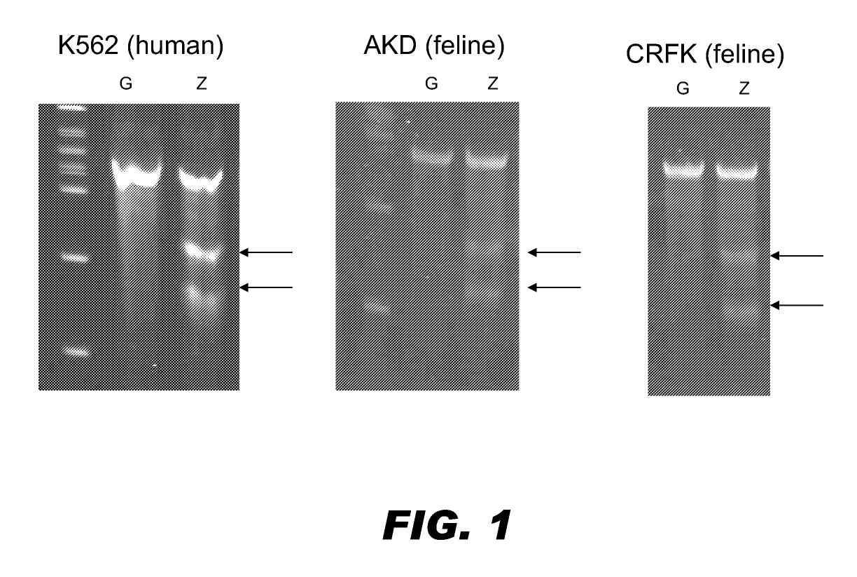

The frequency of ZFN-induced double strand chromosomal breaks was determined using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ. As shown in FIG. 1, the SMAD4 ZFNs (19160/19159) cleaved the SMAD4 locus in human and cat cells.

### Example 2

### Genome Editing of SMAD4 in Cat Embryos

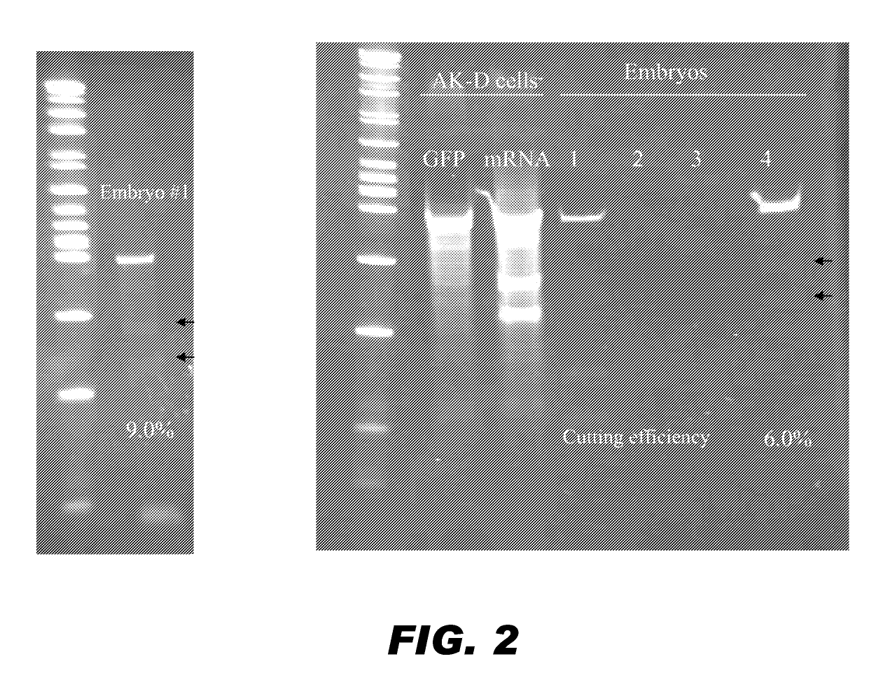

Cat embryos were harvested using standard procedures and injected with capped, polyadenylated mRNA encoding SMAD4 ZFNs (19160/19159) using techniques substantially similar to those described by Geurts et al. Science (2009) 325:433, which is incorporated by reference herein in its entirety. The cat embryos were at the 2-4 cell stage when microinjected. Control embryos were injected with 0.1 mM EDTA. The frequency of cutting was estimated using the Cel-1 assay as described in Example 1. As illustrated in FIG. 2, the cutting efficiency was estimated to be about 6-9%.

TABLE 2 presents the development of the embryos following microinjection. About 19% (3/16) of the embryos injected with a small volume of SMAD4 ZFN mRNA developed to the blastula stage, and 50% (8/16) of the control embryos injected with EDTA developed to the blastula stage.

TABLE 2
Embryo development

| | | | | | | Day 5 | | Day 7/8 |
|---|---|---|---|---|---|---|---|---|
| | | | No. oocytes | Day 2 | | Degenerated/ | Morula/ | Blastocysts/ |
| | No. | No. oocytes | IVF or | | Cleaved | Non-further- | cleaved | cleaved |
| Treatment | replicates | collected | injected | Degenerated | (2-4 cells) | cleavage | embryos | embryos |
| IVF | N = 2 | 58 | 54 | 1/54 | 34/54 | 0/34 | 34/34 | 33-34 |
| (control) | | | | (1.8%) | (62.9%) | (0%) | (100%) | (97%) |
| 0.1 mM | N = 1 | 18 | 16 | 0/16 | 15/16 | 1/15 | 14/15 | 8/15 |
| EDTA | | | | (0%) | (93.7%) | (6.6%) | (93.3%) | (53.3%) |
| ZFN- | Higher | 44 | 36 | 2/36 | 34/36 | 29/31 | 2/31 | 0/31 |
| SMAD- | volume | | | (5.5%) | (86.1%) | (93.5%) | (6.4%) | (0%) |
| 4 | N = 1 | | | | | | | |
| (10 ng) | Smaller | 20 | 16 | 3/16 | 5/16 | 2/5 | 3/5 | 3/5 |
| | volume | | | (18.7%) | (31.2%) | (40.0%) | (60%) | (60%) |
| | N + 1 | | | | | | | |

### Example 3

### Genome Editing of Fel d1 in Cat Cells

ZFNs were designed to target different regions of the Fel d1 chromosomal sequence in cat (see Geurts et al. (2009) supra). The ZFNs targeted chain 1-exon 1, chain 1-exon 2, or chain 2-exon 2 of Fel d1. The amino acid sequence and DNA binding site of each ZFN are shown in TABLE 3.

TABLE 3
Fel d1 ZFNs

| | | SEQ | DNA binding site | SEQ |
|---|---|---|---|---|
| Name | ZFN protein sequence | ID NO: | (Contact sites in uppercase) | ID NO: |
| 17 | VPAAMAERPFQCRICMRNFSRSDHL | 3 | acAGTAGGGCAGGGTGGgagggctgcgt | 11 |
| (ch1, ex1) | STHIRTHTGEKPFACDICGRKFARS | | | |
| | AHLSRHTKIHTGSQKPFQCRICMRN | | | |
| | FSQSGSLTRHIRTHTGEKPFACDIC | | | |
| | GRKFARSDHLTQHTKIHTGEKPFQC | | | |
| | RICMRKFALKQHLNEHTKIHLRGS | | | |
| | VPAAMAERPFQCRICMRNFSRSDNL | 4 | ggCCACAGCAGGTATAAAAGggttccag | 12 |
| 18 | SAHIRTHTGEKPFACDICGRKFAQS | | | |
| (ch1, ex1) | ANRIKHTKIHTGSQKPFQCRICMRN | | | |
| | FSQSGALARHIRTHTGEKPFACDIC | | | |
| | GRKFARSDNLREHTKIHTGSQKPFQ | | | |
| | CRICMRNFSRSDHLSEHIRTHTGEK | | | |
| | PFACDICGRKFAQSATRKKHTKIHL | | | |
| | RGS | | | |
| 7 | VPAAMAERPFQCRICMRNFSQSGHL | 5 | tcGTCGGGggTTCCCGTCAGGAataggt | 13 |
| (ch1, ex2) | ARHIRTHTGEKPFACDICGRKFAQS | | | |
| | ADRTKHTKIHTGSQKPFQCRICMRN | | | |
| | FSRSDTLSEHIRTHTGEKPFACDIC | | | |
| | GRKFANRRGRWSHTKIHTPNPHRRT | | | |
| | DPSHKPFQCRICMRNFSRSDHLSRH | | | |
| | IRTHTGEKPFACDICGRKFADPSYL | | | |
| | PRHTKIHLRGS | | | |
| 9 | VPAAMAERPFQCRICMRNFSRSDSL | 6 | atGTTGAGCAAGTGgcacaatacaatgc | 14 |
| (ch1, ex2) | SVHIRTHTGEKPFACDICGRKFAQN | | | |
| | ANRKTHTKIHTGSQKPFQCRICMRN | | | |
| | FSRSANLARHIRTHTGEKPFACDIC | | | |
| | GRKFATSGSLTRHTKIHLRGS | | | |
| 12 | VPAAMAERPFQCRICMRNFSRSDTL | 7 | aaGAGTCCGTTcTCCACGtagcaatcct | 15 |
| (ch2, ex2) | SAHIRTHTGEKPFACDICGRKFADK | | | |
| | RTRTTHTKIHTHPRAPIPKPFQCRI | | | |
| | CMRNFSTSGSLSRHIRTHTGEKPFA | | | |
| | CDICGRKFADSSDRKKHTKIHTGEK | | | |
| | PFQCRICMRKFARSDNLTRHTKIHL | | | |
| | RGS | | | |
| 13 | VPAAMAERPFQCRICMRNFSRSDTL | 8 | ccAGGGTCtTGGATGGACTAGtcatggt | 16 |
| (ch2, ex2) | SAHIRTHTGEKPFACDICGRKFADK | | | |
| | RTRTTHTKIHTHPRAPIPKPFQCRI | | | |
| | CMRNFSTSGSLSRHIRTHTGEKPFA | | | |
| | CDICGRKFADSSDRKKHTKIHTGEK | | | |
| | PFQCRICMRKFARSDNLTRHTKIHL | | | |
| | RGS | | | |

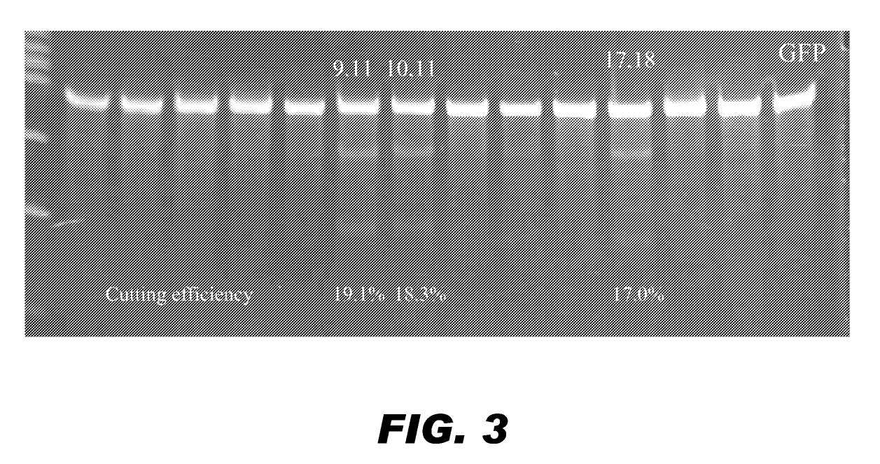

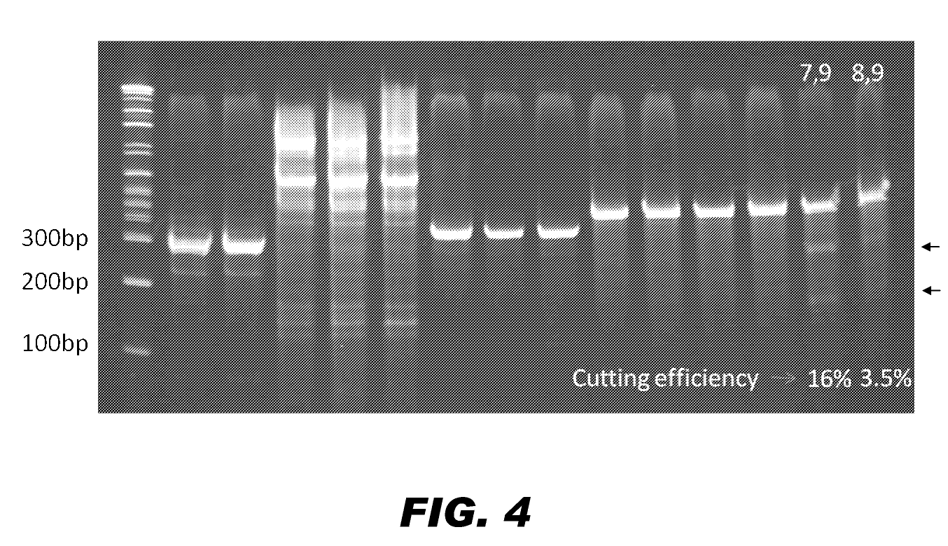

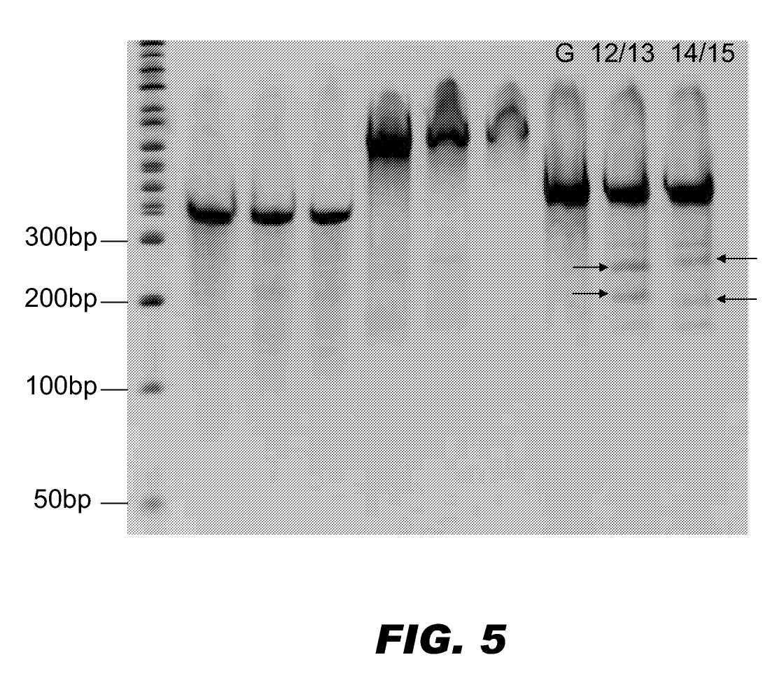

Feline AKD cells were transfected with mRNA encoding Fel d1 ZFNs (17/18), which target exon 1 of chain 1; Fel d1 ZFNs (7/9), which target exon 2 of chain 1, or Fel d1 ZFNs (12/13), which target exon 2 of chain 2. The efficiency of ZFN-mediated cutting was estimated using the Cel-1 assay as described above. The cutting efficiency of the 17/18 Fel d1 ZFN pair was estimated to be about 17% (see FIG. 3). The 7/9 Fel d1 ZFN pair cleaved chain 1, exon 2 with an efficiency of about 16% (see FIG. 4). FIG. 5 illustrates that chain 2, exon 2 was cleaved by the 12/13 Fel d1 ZFN pair.

### Example 4

### Genome Editing of Fel d1 in Cat Embryos

To facilitate inactivation of the Fel d1 locus, cat embryos were treated with two pairs of Fel d1 ZFNs. One pair (17/18) targeted chain 1-exon 1 and the other pair (12/13) targeted chain 2-exon 2. Because of the genomic organization of Fel d1 locus, the coding region for chain 2 (which is transcribed from the “lower” strand) is located about 4000 bp upstream of the coding region for chain 1 (which is transcribed from the “upper” strand). Thus, it was hypothesized that editing events at two separate locations may mediate a large deletion from the Fel d1 locus. Cat embryos were co-injected with capped, polyadenylated mRNAs encoding the pairs of ZFNs essentially as described above in Example 2. TABLE 4 presents the development of the embryos following microinjection. Embryos injected with the higher concentration Fel d1 ZFNs had a higher survival rate than those injected with the lower concentration.

TABLE 4
Embryo development.

| No total | | | No. of | Day 2 | | Day 5 | | Day 7/8 |
|---|---|---|---|---|---|---|---|---|
| oocytes | | | fertilized | | Cleaved | Degenerated/ | Morula/ | Blastocysts/ |
| collected | | No. | oocytes | | (2-4 | No further | cleaved | cleaved |
| (n = cats) | Treatment | replicates | injected | Degenerated | cells) | cleavages | embryos | embryos |
| 100 | IVF | | 5 | | 5 | | 5 | 4 |
| (n = 4) | (Control) | | | | | | | (80%) |
| | EDTA | | | | | | | |
| | control | | | | | | | |
| | Feld1 | 2 | 31 | 4 | 18 | 3 | 15 | 4 |
| | 40 ng/uL | | | | | | | (22%) |
| | Feld1 | 1 | 27 | 1 | 14 | 3 | 11 | 1 |
| | 20 ng/uL | | | | | | | (7.1%) |

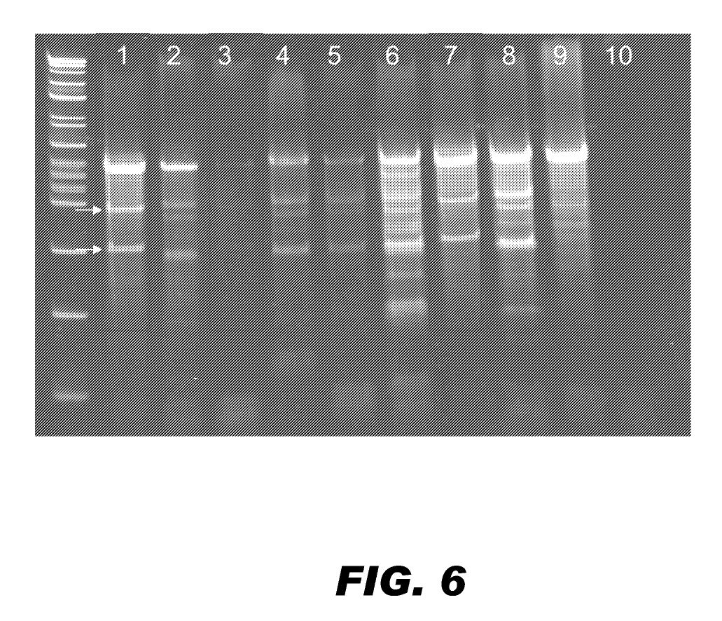

On day 8, the control and experimental embryos were harvested for analysis. Control blastocysts contained about 150-300 cells, experimental blastocysts contained about 70-100 cells, and experimental morula contained about 16-30 cells. DNA of individual embryos was extracted using standard procedures, and subjected to Cel-1 analysis (see FIG. 6). Samples in lanes 1, 3, and 7 displayed the expected Cel-1 digestion products. Sequence analysis revealed that extra bands in other lanes (including the control lane, 6) were due to nearby SNPs.

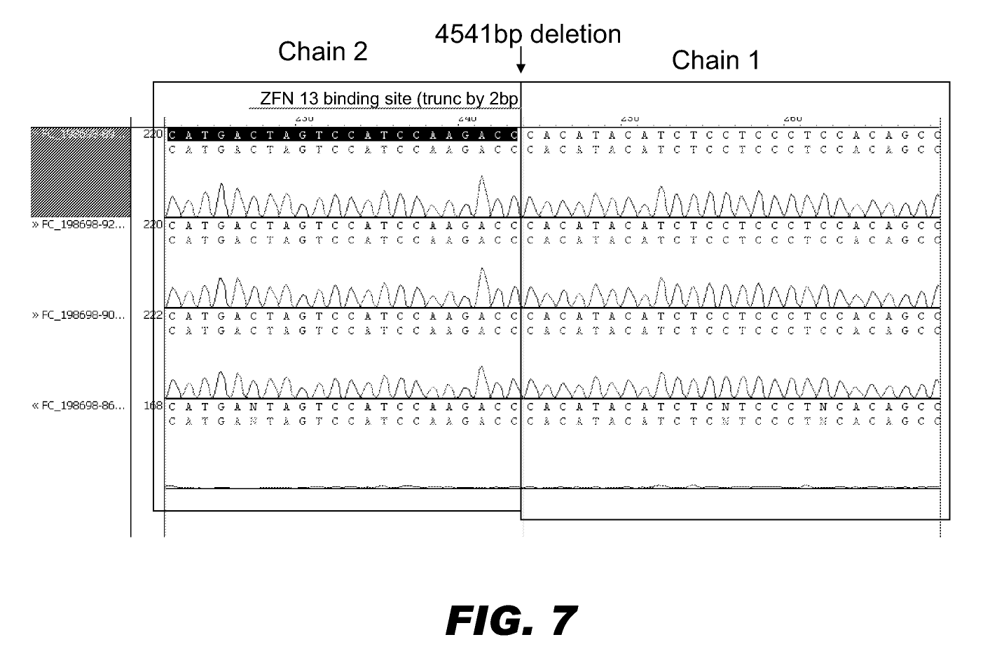

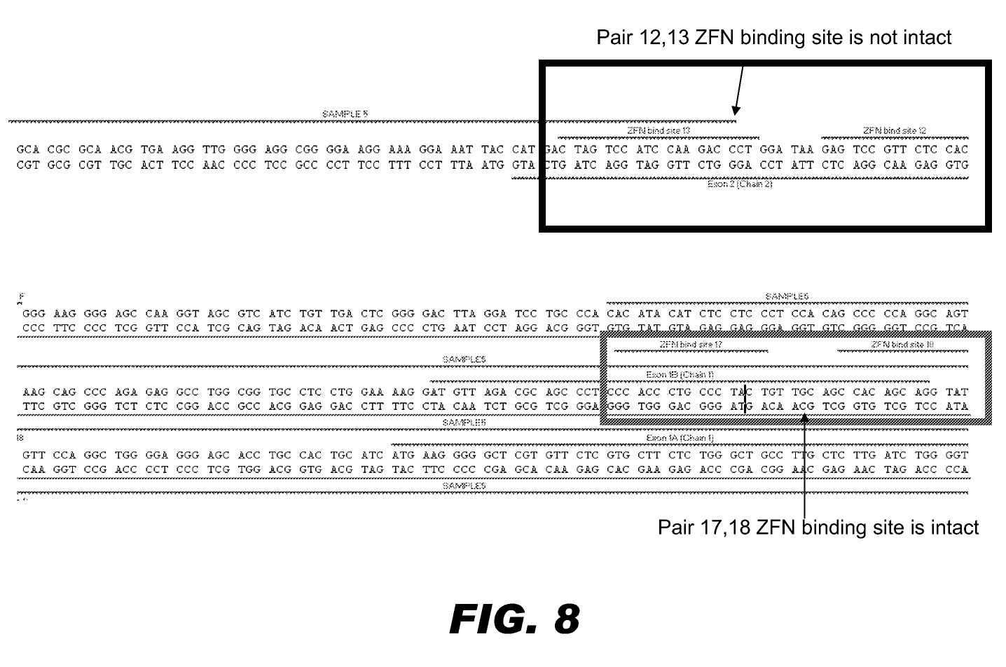

To further analyze the edited Fel d1 loci, the targeted region was PCR amplified and sequenced using standard methods. Sequence analysis confirmed that sample #5 had a 4541 bp deletion between the coding regions for chain 2 and chain 1 (see FIG. 7). In particular, the binding site for ZFN 13 was truncated by 2 bp and the binding for ZFN 12 was deleted along with additional downstream sequence. Surprisingly, the binding site for the 17/18 pair was intact, indicating that the deletion was a result of cleavage by the 12/13 ZFN pair (see FIG. 8).

### Example 5

### Genome Editing of Cauxin in Cat Cells

Pairs of ZFNs that target regions of the cauxin locus were designed and tested in cat cells as detailed above. TABLE 5 presents the amino acid sequence of the zinc finger helices and DNA binding site of each active ZFN.

TABLE 5
Cauxin ZFNs

| | | | DNA binding site | |
|---|---|---|---|---|
| | | SEQ | (Contact sites in | SEQ |
| Name | Sequence of Zinc Finger Helices | ID NO: | uppercase) | ID NO: |
| 1 | QSGNLAR LAYDRRK RSDTLSE QSSHLAR | 19 | atCCGGCTGGACCGTCTG | 27 |
| (exon 1) | QSSDLSR RRDTLLD | | AActcctagc | |
| 2 | QSGDLTR NKHHRNR RSDALAR TSGNLTR | 20 | agTGGGATGTGGGTGCAc | 28 |
| (exon 1) | RRYYLRL | | ccaggccgga | |
| 9 | RSDNLAR WRGDRVK DRSHLAR QSSDLSR | 21 | caGCAGCTGGCCCTGAGg | 29 |
| (exon 2) | QSGDLTR | | ggacacacag | |
| 10 | RSDNLSE SSRNLAS RSANLAR RSDNLTR | 22 | tgCACCAGtGAGGAGCAC | 30 |
| (exon 2) | RSDNLSE SSRNLAS | | CAGgctggga | |
| 17 | DSSDRKK QSSDLSR YHWYLKK RSDHLSQ | 23 | taCAGTGGTTTGCTTCCc | 31 |
| (exon 2) | TSANRTT | | ccggacccat | |
| 18 | QSGNLAR WLSSLGI DRSDLSR LRFNLRN | 24 | ggGAAGCAcCATGCCTGT | 32 |
| (exon 2) | QSGDLTR QSGNLAR | | GAAcatgttc | |
| 29 | DRSNLSR DAFTRTR RSDNLSV ERGTLAR | 25 | agGCAGCCAAGGCGGACc | 33 |
| (exon 4) | QSGDLTR | | catcgaagga | |
| 30 | TNHGLNE TSSNLSR QSSDLSR HKYHLRS | 26 | gaGGACGTGCTGATCGTg | 34 |
| (exon 4) | QSGHLSR | | actacccagt | |

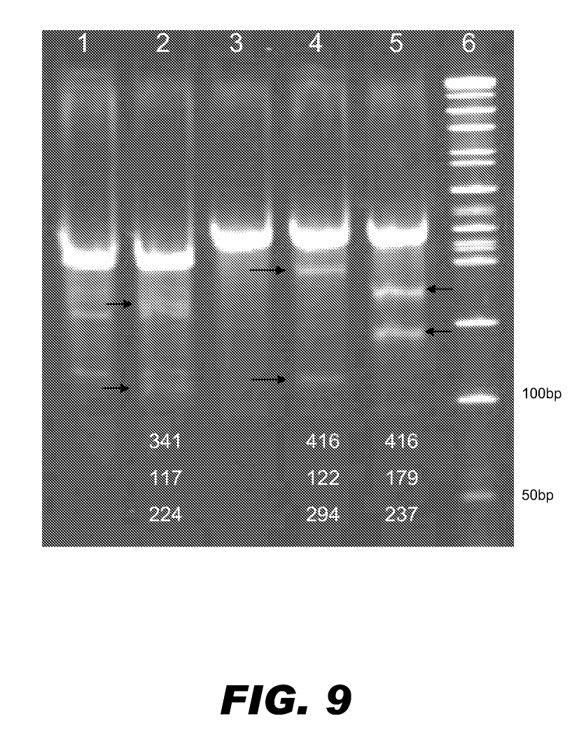

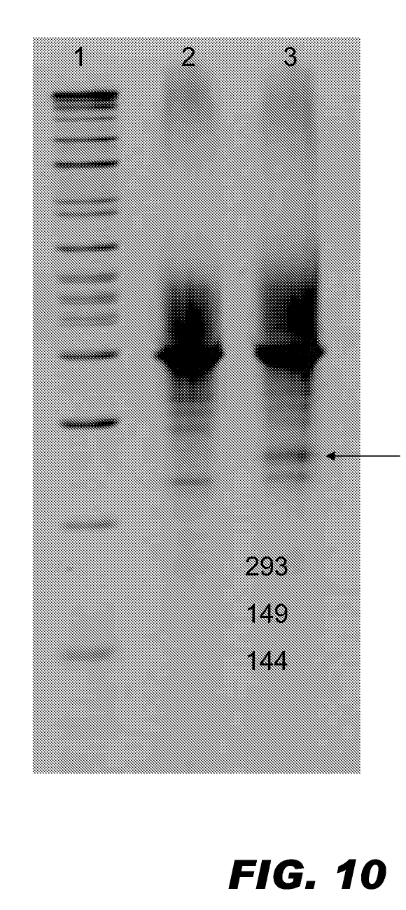

FIG. 9 presents results from a Cel-1 assay in which cleavage of the cauxin locus by the 1/2 pair, the 9/10 pair, and the 17/18 ZFN pairs were confirmed. FIG. 10 Illustrates cleavage of the cauxin locus by the 29/30 ZFN pair.

What is claimed is:

1. A genetically modified feline comprising at least one edited chromosomal sequence.

2. The genetically modified feline of claim 1, wherein the edited chromosomal sequence is inactivated, is modified, or has an integrated sequence.

3. The genetically modified feline of claim 1, wherein the edited chromosomal sequence encodes a protein chosen from Felis domesticus 1 (Fel d1), tyrosinase (TYR), tyrosinase-related protein 1 (TYRP1), augoti signaling protein (ASIP), melanophilin (MLPH), fibroblast growth factor 5 (FGF5), cauxin, and combinations thereof.

4. The genetically modified feline of claim 3, wherein the protein is Fel d1, and the edited chromosomal sequence comprises at least one mutation in at least one of its chains such that the sequence is inactivated and no functional protein is produced.

5. The genetically modified feline of claim 4, wherein the feline is hypoallergenic.

6. The genetically modified feline of claim 3, wherein the protein is TYR, and the edited chromosomal sequence comprises at least one mutation such that the sequence is modified and the expressed protein comprises at least one amino acid change.

7. The genetically modified feline of claim 6, wherein the feline has a different coat color, coat pattern, and/or eye color than a feline in which the chromosomal region is not edited.

8. The genetically modified feline of claim 6, wherein the feline comprises a Burmese or Siamese phenotype.

9. The genetically modified feline of claim 3, wherein the protein is TYRP1, ASIP, or MLPH, and the edited chromosomal sequence comprises at least one mutation such that the sequence is modified or inactivated.

10. The genetically modified feline of claim 9, wherein the feline has a different coat color, coat pattern, and/or eye color than a feline in which the chromosomal region is not edited.

11. The genetically modified feline of claim 3, wherein the protein is FGF5, and the edited chromosomal sequence comprises at least one mutation such that the sequence is modified and the expressed protein comprises at least one amino acid change.

12. The genetically modified feline of claim 11, wherein the feline has a different hair phenotype than a feline in which the chromosomal sequence is not edited.

13. The genetically modified feline of claim 3, wherein the protein is cauxin, and the edited chromosomal sequence comprises at least one mutation such that the sequence is inactivated and no functional protein is produced.

14. The genetically modified feline of claim 13, wherein the feline does not excrete felinine or felinine-related compounds.

15. The genetically modified feline of claim 1, wherein the feline is heterozygous or homozygous for the edited chromosomal sequence.

16. The genetically modified feline of claim 1, wherein the feline is a domestic cat.

17. The genetically modified feline of claim 1, wherein the feline is an embryo, a kitten, a juvenile, or an adult.

18. A feline embryo, the embryo comprising at least one RNA molecule encoding a zinc finger nuclease that is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

19. The feline embryo of claim 18, wherein the chromosomal sequence encodes a protein chosen from Felis domesticus 1 (Fel d1), tyrosinase (TYR), tyrosinase-related protein 1 (TYRP1), augoti signaling protein (ASIP), melanophilin (MLPH), fibroblast growth factor 5 (FGF5), cauxin, and combinations thereof.

20. A genetically modified feline cell comprising at least one edited chromosomal sequence.

21. The genetically modified feline cell of claim 20, wherein the edited chromosomal sequence encodes a protein chosen from Felis domesticus 1 (Fel d1), tyrosinase (TYR), tyrosinase-related protein 1 (TYRP1), augoti signaling protein (ASIP), melanophilin (MLPH), fibroblast growth factor 5 (FGF5), cauxin, and combinations thereof.

22. The genetically modified feline cell of claim 20, wherein the protein is chosen from Fel d1, TYRP1, ASIP, MLPH, FGF5, cauxin, and combinations thereof, and the edited chromosomal sequence comprises at least one mutation such that the sequence is inactivated and no functional protein is produced.

23. The genetically modified feline cell of claim 20, wherein the protein is chosen from TYR, TYRP1, ASIP, MLPH, and FGF5, and combinations thereof, and the edited chromosomal sequence comprises at least one mutation such that the sequence is modified and the expressed protein comprises at least one amino acid change.

24. The genetically modified feline cell of claim 20, wherein the cell is heterozygous or homozygous for the edited chromosomal sequence.

25. The genetically modified feline cell of claim 20, wherein the cell is derived from a domestic cat.

26. A zinc finger nuclease, the zinc finger nuclease comprising a zinc finger DNA binding domain and a cleavage domain, the zinc finger nuclease having an amino acid sequence that has at least about 80% sequence identity to a sequence chosen from SEQ ID NOs:1, 2, 3, 4, 5, 6, 7, and 8, or the zinc finger DNA binding domain helices have an amino acid sequence that is at least about 80% identical to a sequence chosen from SEQ ID NOs:19, 20, 21, 22, 23, 24, 25, and 26.

27. The zinc finger nuclease of claim 26, wherein the sequence identity is at least about 85%, 90%, 95%, or 100%.

28. The zinc finger nuclease of claim 26, wherein the DNA binding domain comprises at least three zinc finger recognition regions.

29. The zinc finger nuclease of claim 26, wherein the DNA binding domain binds a chromosomal sequence having at least about 80% sequence identity to a sequence chosen from SEQ ID NOs:9, 10, 11, 12, 13, 14, 15, 16, 27, 28, 29, 30, 31, 32, 33, and 34.

30. The zinc finger nuclease of claim 36, wherein the sequence identity is at least about 85%, 90%, 95%, or 100%.

31. The zinc finger nuclease of claim 26, wherein the cleavage domain is a wild-type or an engineered FokI cleavage domain.

32. A nucleic acid sequence bound by a zinc finger nuclease, the nucleic acid sequence having at least 80% sequence identity with a sequence chosen from SEQ ID NOs:9, 10, 11, 12, 13, 14, 15, 16, 27, 28, 29, 30, 31, 32, 33, and 34.

33. The nucleic acid sequence of claim 32, wherein the sequence identity is at least about 85%, 90%, 95%, or 100%.


SEQUENCE LISTING (34 sequences)

SEQ ID NO: 1 - protein, 149 aa, Artificial Sequence
  SYNTHESIZED
VPAAMAERPFQCRICMRNFSRSDNLARHIRTHTGEKPFACDICGRKFAQSSDLRRHTKIHTGGQRPFQCRICMRNFSRSDTLSQHIRTHTGEKPFACDICGRKFADRSARTRHTKIHTGEKPFQCRICMRKFAQSSDLRRHTKIHLRGS

SEQ ID NO: 2 - protein, 181 aa, Artificial Sequence
  SYNTHESIZED
VPAAMAERPFQCRICMRNFSERGTLARHIRTHTGEKPFACDICGRKFAQSADRTKHTKIHTGGQRPFQCRICMRNFSRSDHLSTHIRTHTGEKPFACDICGRKFADNANRTKHTKIHTGGGGSQKPFQCRICMRNFSQSSNLARHIRTHTGEKPFACDICGRKFARSDALTQHTKIHLRGS

SEQ ID NO: 3 - protein, 149 aa, Artificial Sequence
  SYNTHESIZED
VPAAMAERPFQCRICMRNFSRSDHLSTHIRTHTGEKPFACDICGRKFARSAHLSRHTKIHTGSQKPFQCRICMRNFSQSGSLTRHIRTHTGEKPFACDICGRKFARSDHLTQHTKIHTGEKPFQCRICMRKFALKQHLNEHTKIHLRGS

SEQ ID NO: 4 - protein, 178 aa, Artificial Sequence
  SYNTHESIZED
VPAAMAERPFQCRICMRNFSRSDNLSAHIRTHTGEKPFACDICGRKFAQSANRIKHTKIHTGSQKPFQCRICMRNFSQSGALARHIRTHTGEKPFACDICGRKFARSDNLREHTKIHTGSQKPFQCRICMRNFSRSDHLSEHIRTHTGEKPFACDICGRKFAQSATRKKHTKIHLRGS

SEQ ID NO: 5 - protein, 186 aa, Artificial Sequence
  SYNTHESIZED
VPAAMAERPFQCRICMRNFSQSGHLARHIRTHTGEKPFACDICGRKFAQSADRTKHTKIHTGSQKPFQCRICMRNFSRSDTLSEHIRTHTGEKPFACDICGRKFANRRGRWSHTKIHTPNPHRRTDPSHKPFQCRICMRNFSRSDHLSRHIRTHTGEKPFACDICGRKFADPSYLPRHTKIHLRGS

SEQ ID NO: 6 - protein, 121 aa, Artificial Sequence
  SYNTHESIZED
VPAAMAERPFQCRICMRNFSRSDSLSVHIRTHTGEKPFACDICGRKFAQNANRKTHTKIHTGSQKPFQCRICMRNFSRSANLARHIRTHTGEKPFACDICGRKFATSGSLTRHTKIHLRGS

SEQ ID NO: 7 - protein, 153 aa, Artificial Sequence
  SYNTHESIZED
VPAAMAERPFQCRICMRNFSRSDTLSAHIRTHTGEKPFACDICGRKFADKRTRTTHTKIHTHPRAPIPKPFQCRICMRNFSTSGSLSRHIRTHTGEKPFACDICGRKFADSSDRKKHTKIHTGEKPFQCRICMRKFARSDNLTRHTKIHLRGS

SEQ ID NO: 8 - protein, 153 aa, Artificial Sequence; identical to SEQ ID NO: 7
  SYNTHESIZED

SEQ ID NO: 9 - DNA, 28 bp, Felis catus
ctgctgtcctggctgaggccctgatgct

SEQ ID NO: 10 - DNA, 28 bp, Felis catus
gaatggatttactggtcagccagctact

SEQ ID NO: 11 - DNA, 28 bp, Felis catus
acagtagggcagggtgggagggctgcgt

SEQ ID NO: 12 - DNA, 28 bp, Felis catus
ggccacagcaggtataaaagggttccag

SEQ ID NO: 13 - DNA, 28 bp, Felis catus
tcgtcgggggttcccgtcaggaataggt

SEQ ID NO: 14 - DNA, 28 bp, Felis catus
atgttgagcaagtggcacaatacaatgc

SEQ ID NO: 15 - DNA, 28 bp, Felis catus
aagagtccgttctccacgtagcaatcct

SEQ ID NO: 16 - DNA, 28 bp, Felis catus
ccagggtcttggatggactagtcatggt

SEQ ID NO: 17 - DNA, 192 bp, Felis catus
  misc_feature (150)..(150): n is a, c, g, or t
catgactagtccatccaagacccacatacatctcctccctccacagcccatgactagtccatccaagacccacatacatctcctccctccacagcccatgactagtccatccaagacccacatacatctcctccctccacagcccatgantagtccatccaagacccacatacatctcntccctncacagcc

SEQ ID NO: 18 - DNA, 372 bp, Felis catus
gcacgcgcaacgtgaaggttggggaggcggggaaggaaaggaaattaccatgactagtccatccaagaccctggataagagtccgttctccacgggaaggggagccaaggtagcgtcatctgttgactcggggacttaggatcctgcccacacatacatctcctccctccacagcccccaggcagtaagcagcccagagaggcctggcggtgcctcctggaaaaggatgttagacgcagccctcccaccctgccctactgttgcagccacagcaggtatgttccaggctggggagggagcacctgccactgcatcatgaagggggctcgtgttctcgtgcttctctgggctgccttgctcttgatctggggt

SEQ ID NO: 19 - protein, 42 aa, Artificial Sequence
  SYNTHESIZED
QSGNLARLAYDRRKRSDTLSEQSSHLARQSSDLSRRRDTLLD

SEQ ID NO: 20 - protein, 35 aa, Artificial Sequence
  SYNTHESIZED
QSGDLTRNKHHRNRRSDALARTSGNLTRRRYYLRL

SEQ ID NO: 21 - protein, 35 aa, Artificial Sequence
  SYNTHESIZED
RSDNLARWRGDRVKDRSHLARQSSDLSRQSGDLTR

SEQ ID NO: 22 - protein, 42 aa, Artificial Sequence
  SYNTHESIZED
RSDNLSESSRNLASRSANLARRSDNLTRRSDNLSESSRNLAS

SEQ ID NO: 23 - protein, 35 aa, Artificial Sequence
  SYNTHESIZED
DSSDRKKQSSDLSRYHWYLKKRSDHLSQTSANRTT

SEQ ID NO: 24 - protein, 42 aa, Artificial Sequence
  SYNTHESIZED
QSGNLARWLSSLGIDRSDLSRLRFNLRNQSGDLTRQSGNLAR

SEQ ID NO: 25 - protein, 35 aa, Artificial Sequence
  SYNTHESIZED
DRSNLSRDAFTRTRRSDNLSVERGTLARQSGDLTR

SEQ ID NO: 26 - protein, 35 aa, Artificial Sequence
  SYNTHESIZED
TNHGLNETSSNLSRQSSDLSRHKYHLRSQSGHLSR

SEQ ID NO: 27 - DNA, 28 bp, Felis catus
atccggctggaccgtctgaactcctagc

SEQ ID NO: 28 - DNA, 28 bp, Felis catus
agtgggatgtgggtgcacccaggccgga

SEQ ID NO: 29 - DNA, 28 bp, Felis catus
cagcagctggccctgaggggacacacag

SEQ ID NO: 30 - DNA, 28 bp, Felis catus
tgcaccagtgaggagcaccaggctggga

SEQ ID NO: 31 - DNA, 28 bp, Felis catus
tacagtggtttgcttcccccggacccat

SEQ ID NO: 32 - DNA, 28 bp, Felis catus
gggaagcaccatgcctgtgaacatgttc

SEQ ID NO: 33 - DNA, 28 bp, Felis catus
aggcagccaaggcggacccatcgaagga

SEQ ID NO: 34 - DNA, 28 bp, Felis catus
gaggacgtgctgatcgtgactacccagt